(19) United States
(12) Patent Application Publication
(10) Pub. No.: US 2007/0197898 A1
(43) Pub. Date: Aug. 23, 2007

(54) Method for examination of vessels in a patient on the basis of image data recorded by means of a scanner within an examination area

(76) Inventor: Nikolaus Bolle, Baiersdorf (DE)

Correspondence Address: HARNESS, DICKEY & PIERCE, P.L.C., P.O.BOX 8910, RESTON, VA, 20195, US

(21) Appl. No.: 11/653,229

(22) Filed: Jan. 16, 2007

(30) Foreign Application Priority Data
Jan. 17, 2006 (DE) 10 2006 002 259.9

(51) Int. Cl. A61B 5/05

(52) U.S. Cl. 600/407

(57) ABSTRACT

A method is disclosed for examination of vessels in a patient on the basis of image data which is recorded by a scanner within an examination area. In at least one embodiment of the method, a sequence of vessel diameters is determined at different positions along at least one of the vessels on the basis of the image data, and signals, by which changes in the vessel diameter can be perceived in an intuitive manner, are generated on the basis of the sequence of determined vessel diameters.

### Priority Statement

The present application hereby claims priority under 35 U.S.C. §119 on German patent application number DE 10 2006 002 259.9 filed Jan. 17, 2006, the entire contents of which is hereby incorporated herein by reference.

### FIELD

Embodiments of the invention generally relate to a method for examination of vessels in a patient, such as a method for examination of vessels in a patient on the basis of image data which is recorded by a scanner within an examination area, for example.

### BACKGROUND

A stenosis is a vessel constriction, for example a blood-vessel constriction. If a stenosis such as this occurs in the area of the heart, then this can lead to the heart being deprived of oxygen. For the patient, this results in a risk of a cardiac infarct. Stenoses must therefore be identified and treated as early as possible.

If there is a suspicion of a constriction in a blood vessel, image data from an examination area is recorded by means of a scanner, for example by way of a computed-tomography scanner or a magnetic resonance imager, and this is used to generate a slice image or volume image. A doctor who is carrying out the treatment can carry out a visual assessment on the basis of an image such as this, in which the vessels are displayed three-dimensionally, in order to identify those blood vessels which are potentially constricted.

The doctor's assessment is assisted by 3D display software, by which images from a virtual camera within the vessel can be produced along user-defined or calculated paths. The sequence of images produced in this way makes it possible for the doctor to identify a constriction in a vessel by way of a gradual change in the vessel diameter in the sequence of images. However, because of complexity of the image information, there is a risk of the potentially suspicious vessel regions not all being identified by purely visual examination of the images.

### SUMMARY

In at least one embodiment of the present invention, an examination of a vessel is simplified on the basis of the recorded image data within an examination area.

The inventor has found, in at least one embodiment, that the examination of a vessel in a patient can be improved by generating signals as a function of the vessel diameters, by which changes in the vessel diameter can be perceived intuitively in a simple manner, instead of or together with visual examination of the images.

The method for examination of vessels in a patient on the basis of image data recorded via a scanner within an examination area accordingly comprises method steps in which a sequence of vessel diameters is determined at different positions along at least one of the vessels on the basis of the image data, and in which signals by which changes in the vessel diameter can be perceived are generated on the basis of the sequence of determined vessel diameters.

The signals generated on the basis of the sequence of vessel diameters determined allows particularly intuitive perception of changes in the vessel diameter, so that incorrect decisions in the assessment of sickness-related vessel changes can be largely avoided.

The determination of the vessel diameters advantageously includes the calculation of a segmented image which contains only the vessels. It is then possible to mark that vessel in the segmented image for which a sequence of vessel diameters is intended to be determined along a center path. The image data is reduced by the segmentation process to that component of the image information which is relevant for determination of the vessel diameter, so that it is possible to determine the vessel diameters with little numerical complexity.

The vessel diameter is determined by determining a center path in the vessel, with the vessel diameter being calculated from the perpendicular distance between an inner wall of the vessel and the center path. The vessel diameter can be calculated easily using digital image-processing methods, with little numerical complexity, and allows reliable determination of the vessel diameter even if the blood vessel has a highly complex three-dimensional profile.

Vessel diameters are advantageously normalized with respect to the maximum observed vessel diameter before the actual generation of the signals. This has the particular advantage that the signal can be generated for very different vessels using the same signal values and the same signal amplitudes. For example, this means that it is possible to make it possible to perceive constrictions in a blood vessel using the same signals as, for example, in the case of the constrictions in urinary vessels, bile ducts or bowel vessels.

The signals are preferably generated in synchronism with the display of a pass through the vessel. The doctor carrying out the treatment is thus presented with two different information items at the same time for assessment of a stenosis, in whose combination vessel changes can be identified more intuitively and more reliably.

The signals are advantageously generated using a signal value which is proportional to the vessel diameter. This results in the doctor carrying out the treatment being provided in a simple manner with information about the size of the vessel diameter from the single value of a generated signal. Furthermore, he is also informed of any change in the vessel diameter when there is a change in the signal value. The doctor carrying out the treatment can also draw conclusions about the nature of the vessel change from the rate of the signal change and from the information in a signal value rise or fall. For example, a signal value rise can indicate a vessel constriction, and the signal value fall can indicate vessel widening.

In one advantageous refinement of at least one embodiment of the invention, gradients are calculated along the vessel from locally adjacent vessel diameters and the signals are generated with a signal value which is proportional to the respective gradients. Calculation of the gradients has the advantage that only changes in the vessel diameter are converted to signals and are indicated to the doctor carrying out the treatment. No signal is generated if the vessel diameters remain the same.

The gradients are preferably compared with a threshold value, with the signals being generated if the gradient exceeds a threshold value, taking into account the mathematical sign of the gradient. Even healthy vessels have changes in the vessel diameter as a function of the vessel type and as a function of the anatomical context. However, the attention of the doctor carrying out the treatment should in general be directed only at changes in the vessel which indicate sickness or widenings in the vessel which indicate sickness. It is thus advantageous for minor fluctuations in the vessel diameter within the scope of a tolerance range not to generate any signals.

Only changes in the vessel diameter beyond a tolerance range should be perceptible for the doctor carrying out the treatment, by the generation of appropriate signals. This can be done by comparing the gradient with a threshold value before the actual signal generation, in which case the threshold value can be preset, or is stored in a databank as a function of the vessel, by the doctor carrying out the treatment.

Furthermore, the signals are preferably generated taking into account the mathematical sign of the gradient, so that it is possible to tell from the signals whether this relates to widening or narrowing of the vessel.

The signals are preferably acoustic signals. The generation of acoustic signals makes it possible for the doctor carrying out the treatment to perceive changes in the vessel diameter in a particularly intuitive form.

In one advantageous refinement of at least one embodiment, vessel diameters becoming smaller are indicated by audio frequencies becoming higher in order to perceive vessel constrictions. Conversely, it is also advantageously feasible for vessel diameters becoming larger to be indicated by audio frequencies becoming lower, in order to perceive vessels becoming wider.

However, the signals can preferably also be optical signals. Optical signals can, for example, be overlaid directly on those images which are generated by the vessel during a pass along the center path of the vessel. In this case, the signal values are advantageously color values. For example, this means that it is feasible to use a green color value to generate a signal when the changes in the vessel diameter fall within the tolerance range of a healthy vessel. Red color values, in contrast, can be used when a vessel change is sufficiently serious that it must be assumed that there is a constriction or widened area in the vessel indicating sickness.

The image data is advantageously recorded via a computed-tomography scanner. In this case, for example, it is possible to use a contrast agent to make blood vessels visible with particularly high contrast in the image data, thus allowing reliable segmentation of the blood vessels to be carried out.

Furthermore, it is likewise feasible for the image data to be recorded via a magnetic resonance imager. Magnetic resonance imaging has the advantage over the imaging processes in diagnostic radiology that the organ can in many cases be displayed better. This is a result of the different nature of the signal intensity which originates from different soft tissues.

### BRIEF DESCRIPTION OF THE DRAWINGS

Example embodiments of the invention as well as further advantageous refinements of the invention are illustrated in the following schematic drawings, in which:

FIG. 1 shows an illustration, partially in perspective form and partially in the form of a block diagram, of a computed tomography scanner which is suitable for carrying out the method according to an embodiment of the invention for examination of vessels,

FIG. 2 shows a segmented blood vessel which has areas with a constricted and a widened vessel,

FIG. 3 shows a diagram which on the one hand shows the contour of the vessel shown in FIG. 2 in the form of a longitudinal section, and on the other hand shows two signal profiles calculated as a function of the vessel diameter.

### DETAILED DESCRIPTION OF THE EXAMPLE EMBODIMENTS

It will be understood that if an element or layer is referred to as being “on”, “against”, “connected to”, or “coupled to” another element or layer, then it can be directly on, against, connected or coupled to the other element or layer, or intervening elements or layers may be present. In contrast, if an element is referred to as being “directly on”, “directly connected to”, or “directly coupled to” another element or layer, then there are no intervening elements or layers present. Like numbers refer to like elements throughout. As used herein, the term “and/or” includes any and all combinations of one or more of the associated listed items.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

The terminology used herein is for the purpose of describing particular embodiments only and is not intended to be limiting of the present invention. As used herein, the singular forms “a”, “an”, and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described.

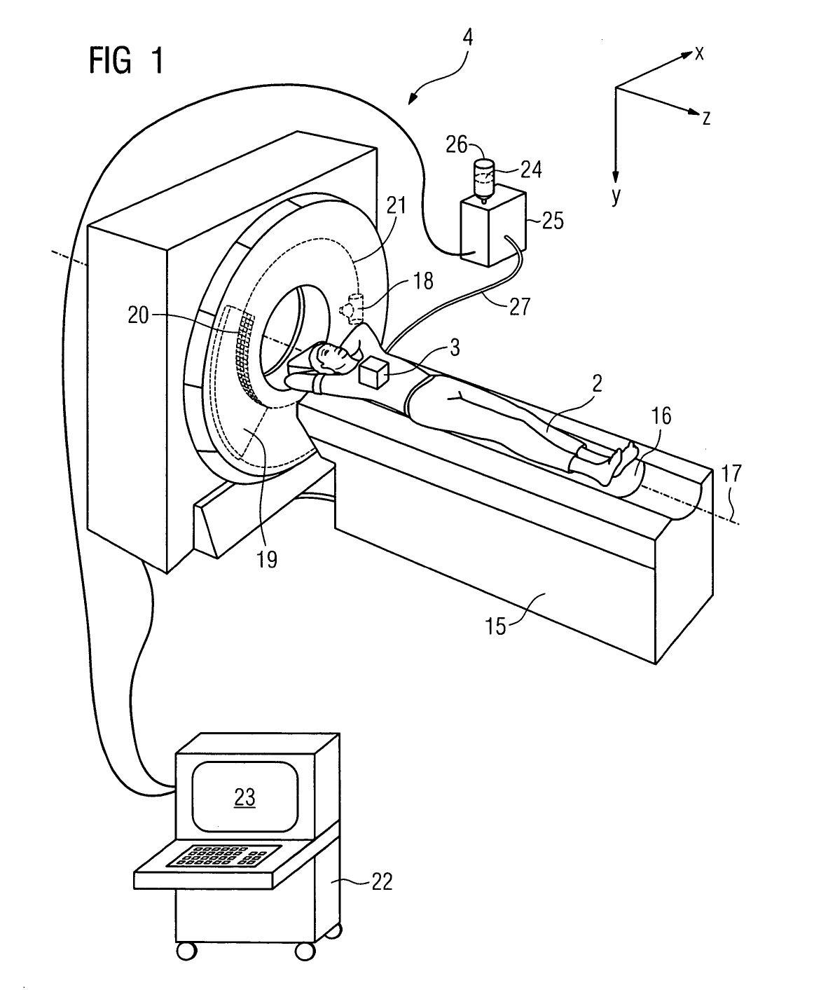

FIG. 1 shows a perspective view of a scanner, in this case a computed-tomography scanner provided with the reference symbol 4, which is suitable for carrying out the method according to an embodiment of the invention for examination of vessels 1 in a patient 2.

The computed-tomography scanner 4 has a supporting apparatus 15 with a moving table top 16, on which the patient 2 can be placed. The table top 16 can be moved in the direction of the rotation axis 17 so that an examination area 3 associated with the patient 2 can be moved through an opening in the housing of the computed-tomography scanner 4 into the measurement area of a recording system 18, 19. The patient 2 and the recording system 18, 19 can in this way be moved relative to one another in the direction of the rotation axis 17, so that different scanning positions can be assumed.

For projection recording, the recording system 18; 19 has an emitter 18 in the form of an X-ray tube and a detector 19 arranged opposite it, with the detector 19 being curved and having a plurality of detector elements 20 arranged to form detector rows. The emitter 18 produces radiation in the form of a fan-shaped X-ray beam, which passes through the measurement area and then strikes the detector elements 20 in the detector 19. The detector elements 20 produce an attenuation value which is dependent on the attenuation of the X-ray radiation passing through the measurement area. By way of example, the X-ray radiation is converted to attenuation values by use of a photodiode, which is optically coupled to a scintillator, or by means of a direct-conversion semiconductor. The detector 19 in this way produces a set of attenuation values, which are also referred to as a projection.

The recording system 18, 19 is arranged on a gantry 21 such that it can rotate, so that projections can be recorded from different projection directions. Depending on the selected operating mode for the computed-tomography scanner 4, the scanning is carried out with a fixed set projection direction or a variable projection direction, with the scanning position at the same time in a fixed setting or variable setting. By way of example, projections are recorded from a large number of different projection directions at different positions along the rotation axis 17 or along the patient 2 by rotation of the gantry 21 with the patient 2 at the same time being moved forward continuously in the direction of the rotation axis 17. The recording-system projections obtained in this way by means of a spiral scan are transmitted to a computation unit 22 and are converted to image data, in which case the image data may, for example, be a slice image or a volume image. The slice image or volume image is then displayed on a display unit 23.

In order to examine vessels, for example a blood vessel, a contrast agent 24 can be injected into the patient 2, by means of a contrast agent appliance 25, in order to increase the visible contrast between the soft tissue parts, if required. The contrast agent 24 is pumped in an automated form, on a time-controlled basis, from a supply container 26 via a flexible contrast-agent tube 27 into a vein in the patient 2, using a variable amount of the contrast agent and an adjustable flow rate.

In comparison to the other soft tissue parts, the contrast agent 24 absorbs more X-ray radiation. The blood vessels through which the contrast agent 24 is passing can be seen by low grey-scale areas in the image that is produced in the grey-scale image of the slice image or volume image, in comparison to the surrounding soft-tissue parts.

A 3D display program assists the doctor to assess the vessel states, with this program being implemented in the computation unit 22 and accessing the image data recorded by the computed-tomography scanner 4. The 3D display program makes it possible to produce images within an examination area 3, with these images being generated by way of a virtual camera which can be navigated such that it moves freely or along a previously calculated path in the examination area. This makes it possible, for example, for the doctor carrying out the treatment to visually inspect the vessels along a calculated center path.

Furthermore, the computation unit 22 has an integrated software module by which signals can be generated which indicate a change in the vessel diameter. A sequence of vessel diameters is determined for this purpose, based on the image data, at different positions along at least one of the vessels, with signals by means of which changes in the vessel diameter can be perceived then being generated on the basis of the sequence of vessel diameters determined.

The signals may be either optical or acoustic signals. This ensures that changes in the vessel can be perceived intuitively by the doctor carrying out the treatment.

The acoustic signals, for example, can be generated in parallel with the display of the image information relating to the vessel. Acoustic signals offer the advantage of very intuitive perception of vessel changes by the doctor carrying out the treatment. For example, it is thus feasible for audio frequencies becoming higher to be used to indicate perception of vessel constriction when the vessel diameters become smaller. Conversely, of course, it would also be feasible for audio frequencies becoming lower to be used to indicate vessel widening when the vessel diameters become larger.

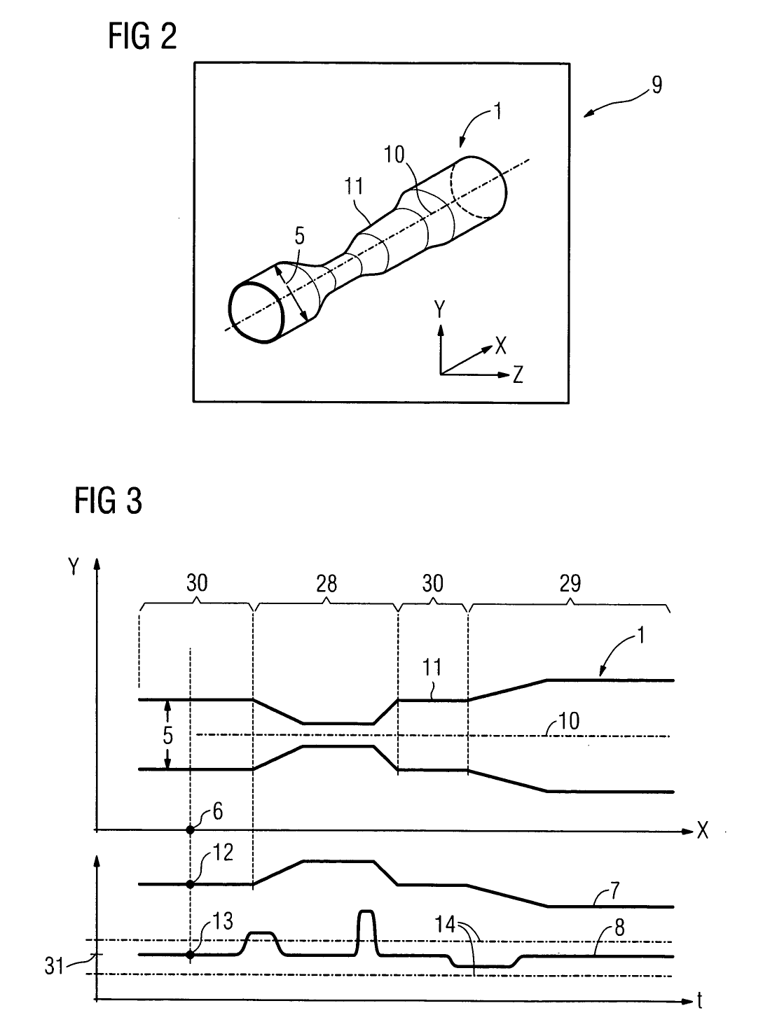

A sequence of vessel diameters along a vessel can be determined particularly easily by segmentation of the vessels in the image data. By way of example, FIG. 2 shows a segmented image 9 with a vessel 1, with the calculated center path 10 through the vessel 1 being shown by a dashed line. The perpendicular distance between the center path 10 and the surface or inner wall 11 of the segmented object corresponds to the radius of the vessel, from which the vessel diameter 5 is determined. For example, it is possible to use segmentation methods in which the areas which are relevant in the image are extracted by means of a threshold-value comparison process. Segmentation processes such as these generally also include morphological operators in order to eliminate image disturbances which are not caused by the vessels 1.

The center path 10 is then calculated on the basis of the segmented image 9. So-called skeletoning processes are used for this purpose, in which, starting from the segmented image 9, the segmented objects are investigated step-by-step starting from the surface until a skeleton structure with a width of 1 pixel remains. This structure corresponds largely to the center path 10 through the vessel 1, along which the sequence of vessel diameters 5 is determined.

In order to allow the numerical complexity to be reduced to the minimum necessary for examination of the vessels, the doctor carrying out the treatment expediently selects individual vessels 1 which are intended to be examined, at the start of the segmentation process. The selection of vessels 1 such as these can be carried out, for example, on the basis of the visually displayed image data, for example within a slice image or volume image. The marking process can be carried out, for example, by means of an input unit, for example a mouse, which is connected to the computation unit 22.

The vessel diameter 5 may, for example, be indicated in units of pixels and is defined such that the number of pixels between the center path 10 and the surface or the inner wall 11 of the segmented object is determined. This can once again be done, for example, with the aid of morphological operators, in which the number of iterations starting from the center path are counted which are necessary for image expansion in order to move from the center path 10 to the surface of the segmented object. The morphological operation is in this case carried out on a directionally selective basis, perpendicular to the center path 10.

FIG. 3 shows the relationship between a vessel change and the generation of the signals 7, 8 in the form of a diagram. The upper part of the diagram shows a longitudinal section through the vessel 1 which was segmented in the illustration in FIG. 2. On the one hand, the vessel 1 has an area with a vessel constriction 28, and on the other hand has an area with vessel widening 29. Two signal profiles are shown in the lower part of the diagram, which are generated to form a sequence of vessel diameters for the vessel 1 shown above. The y axis corresponds to the audio frequency, and the x axis corresponds to the time profile of the signal 7;8. One and only one signal value 12 is associated with each vessel diameter 5 in the case of the upper signal 7. Vessel constrictions 28 can be perceived by the audio frequency of the generated signal 7 becoming higher as the vessel diameters become smaller. Conversely, vessel widenings 29, as illustrated on the diagram, can be perceived by the audio frequency of the generated signal decreasing as the vessel diameter becomes larger.

The second signal 8 was determined on the basis of calculated gradients of the vessel diameter 5. The gradients are in this case determined from the changes in the vessel diameter 5 in the direction of the longitudinal axis of the vessel 1. Areas in which the vessel diameters 30 are constant have a gradient magnitude of zero and, for example, this is associated with a specific basic audio frequency 31. Starting from this basic audio frequency 31, the audio frequency can be increased if the gradient is negative, but the signal amplitude of the generated signal being chosen as a function of the observed magnitude of the gradient. Conversely, it is also feasible for the audio frequency to be correspondingly reduced, starting from the basic audio frequency 31 in the case of positive gradients in the sequence of vessel diameters, with the signal amplitude likewise being dependent on the magnitude of the gradient.

The signal amplitude of the generated signal in the illustrated example is likewise set to zero for gradients with a gradient magnitude of zero, so that the doctor carrying out the treatment can perceive a signal only when there is a change in the vessel. The different treatment of negative and positive gradients makes it possible to intuitively inform the doctor about the geometric change in the vessel by a rise or a fall in the audio frequency. The change in the signal amplitude and the change in the audio frequency may, for example, be directly related to the severity of the change, thus additionally improving the intuitive perception of the change.

In addition to the generation of the acoustic signals, it would, however, likewise be possible to generate optical signals as well, with a color value being changed, rather than changing the audio frequency. The optical signals can either be reproduced optically separately by means of a dedicated apparatus, for example an LED, although it would likewise be feasible for the optical signal to be overlaid directly on the displayed images of the vessel as well.

The method described here is, however, not just restricted to the examination of blood vessels, and can also be used in a corresponding manner to examine bile ducts, urinary vessels or bowel vessels.

The method is likewise not dependent on the modality used to generate the image data. For example, it would be feasible for the image data to be recorded using a magnetic resonance imager instead of a computed-tomography scanner. However, it is likewise also possible for the method to use modalities such as PET scanners or ultrasound-based imagers.

At least one embodiment of the invention can be summarized as follows:

At least one embodiment of the invention relates to a method for examination of vessels 1 in a patient 2 on the basis of image data which is recorded by a scanner within an examination area 3, in which a sequence of vessel diameters 5 is determined at different positions 6 along at least one of the vessels 1 on the basis of the image data, and in which signals 7; 8 by means of which changes in the vessel diameter can be perceived in an intuitive manner are generated on the basis of the sequence of determined vessel diameters 5.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for examination of vessels in a patient on the basis of image data recorded by a scanner within an examination area, the method comprising:
determining a sequence of vessel diameters at different positions along at least one of the vessels on the basis of the image data; and
generating signals, by which changes in the vessel diameter are perceivable, on the basis of the sequence of determined vessel diameters.

2. The method as claimed in claim 1, wherein a segmented image which contains only the vessels is calculated in order to determine the vessel diameters, and wherein the vessel is marked in the segmented image.

3. The method as claimed in claim 1, wherein a center path of the vessel is established to determine the vessel diameter, and wherein the vessel diameter is calculated from a perpendicular distance between an inner wall of the vessel and the center path.

4. The method as claimed in claim 1, wherein the vessel diameters are normalized with respect to a maximum observed vessel diameter before the generation of the signals.

5. The method as claimed in claim 1, wherein the signals are generated in synchronism with the display of a pass through the vessel.

6. The method as claimed in claim 1, wherein the signals are generated using a signal value which is proportional to the vessel diameter.

7. The method as claimed in claim 1, wherein gradients are calculated from vessel diameters which are locally adjacent along the vessel, and wherein the signals are generated using a signal value which is proportional to the respective gradient.

8. The method as claimed in claim 7, wherein the gradient magnitudes are compared with a threshold value, and wherein the signals are generated, taking into account the mathematical sign of the gradient, if the gradient magnitude exceeds a threshold value.

9. The method as claimed in claim 1, wherein the signals are acoustic signals.

10. The method as claimed in claim 7, wherein the signal values are audio frequencies.

11. The method as claimed in claim 10, wherein audio frequencies which become higher as the vessel diameters become smaller are indicated in order to perceive vessel constrictions.

12. The method as claimed in claim 1, wherein the signals are optical signals.

13. The method as claimed in claim 7, wherein the signal values are color values.

14. The method as claimed in claim 1, wherein the image data is recorded via a computed-tomography scanner.

15. The method as claimed in claim 1, wherein the image data is recorded via a magnetic resonance imager.

16. The method as claimed in claim 2, wherein a center path of the vessel is established to determine the vessel diameter, and wherein the vessel diameter is calculated from a perpendicular distance between an inner wall of the vessel and the center path.

17. The method as claimed in claim 8, wherein the signal values are audio frequencies.

18. The method as claimed in claim 8, wherein the signal values are color values.

19. A computer readable medium including program segments for, when executed on a computer device, causing the computer device to implement the method of claim 1.

20. A computer readable medium including program segments for, when executed on a computer device of a computed-tomography scanner, causing the computed-tomography scanner to implement the method of claim 1.

21. A computer readable medium including program segments for, when executed on a computer device of a magnetic resonance imager, causing the magnetic resonance imager to implement the method of claim 1.
